(19) United States
(12) Patent Application Publication
(10) Pub. No.: US 2017/0129864 A1
(43) Pub. Date: May 11, 2017

(54) NEW SIGMA-RECEPTORS LIGANDS WITH ANTI-APOPTOTIC AND/OR PRO-APOPTOTIC PROPERTIES, OVER CELLULAR BIOCHEMICAL MECHANISMS WITH NEUROPROTECTIVE, ANTI-CANCER, ANTI-METASTATIC AND ANTI-(CHRONIC) INFLAMMATORY ACTION

(71) Applicant: Alexandre Vamvakides, Ymittos Attikis (GR)

(72) Inventor: Alexandre Vamvakides, Ymittos Attikis (GR)

(73) Assignee: Anavex Life Sciences Corp., Geneva (CH)

(21) Appl. No.: 13/777,471

(22) Filed: Feb. 26, 2013

Related U.S. Application Data
(63) Continuation of application No. 12/522,761, filed on Jul. 10, 2009, now abandoned.

(30) Foreign Application Priority Data
Jan. 17, 2007 (GR) 20070100020

(51) Int. Cl. C07D 307/14

(52) U.S. Cl. CPC C07D 307/14

(57) ABSTRACT

The present invention involves new and original sigma receptors ligands: (Mono-or di-alkylaminoalkyl)-γ-butyrolactones, their analogues aminotetrahydrofuranes, the (1-adamantyl) phenyl(s) alkylamines, the N,N Dialkyl α-[(adamantyl-l)benzyloxy-2] alkylamines and the 3-cyclopentyl adamantyl-amines or alkylamines or alkyl phenylamines, their enantiomers or diastereoisomers and their pharmaceutically acceptable salts, with pro-apoptotic and/or anti-apoptotic properties over cellular biochemical mechanisms, with anti-cancer, anti-metastatic, anti-(chronic) inflammatory, neuro-protective, anticonvulsive, antidepressive and nooanaleptic or sedative action.

### CROSS-REFERENCE TO RELATED APPLICATIONS

This application is a continuation and claims priority to U.S. Ser. No. 12/522,761, filed Jul. 10, 2009, which is a U.S. national phase application under 35 U.S.C. §371 based upon International Application No. PCT/GR2008/000002 filed on Jan. 14, 2008. Additionally, this U.S. national phase application claims the benefit of priority of International Application No. PCT/GR2008/000002 filed on Jan. 14, 2008, and Greece Application No. 20070100020 filed on Jan. 17, 2007. The entire disclosures of the prior applications are incorporated herein by reference. The international application was published on Jul. 24, 2008 under Publication No. WO 2008/087458.

### BACKGROUND OF THE INVENTION

Field of the Invention

The present invention relates to a sigma(σ)-receptor ligands compound with antiapoptotic and/or pro-apoptotic properties for use in connection with exhibiting a multicomponent pharmacological profile with orthosteric, (σ-1 and/or σ-z-induced) allosteric, and (σ-1 and/or σ-2-induced) pseudo-allosteric modulatory actions over functional biochemical reactions.

### SUMMARY OF THE INVENTION

In view of the foregoing disadvantages inherent in the prior art, the present invention provides an improved sigma(σ)-receptor ligands and method for using, and overcomes the above-mentioned disadvantages and drawbacks of the prior art. As such, the general purpose of the present invention, which will be described subsequently in greater detail, is to provide a new and improved sigma(σ)-receptor ligands and method which has all the advantages of the prior art mentioned heretofore and many novel features that result in a sigma(σ)-receptor ligands which is not anticipated, rendered obvious, suggested, or even implied by the prior art, either alone or in any combination thereof.

To attain this, the present invention essentially comprises a method for using sigma(σ)-receptor ligands. The method includes the providing of a composition of sigma(σ)-receptor ligands comprising aminotetrahydrofuranes, their enantiomers or diastereoisomers and their pharmaceutically acceptable salts. Then using the composition for the preparation of pharmaceutical products.

The pharmaceutical products being with anti-cancer, anti-metastatic and anti(chronic)inflammatory action.

The sigma(σ)-receptor ligands may also be selected from (Mono- or Dialkylaminoalkyl)-y-butyrolactones), their analogues, (1-adamantyl)phenyl(s) alkylamines, N,N di-alkyl a-[(adamantyl-1) benzyloxy-2] alkylamines and 3-cyclopentyl adamantyl-amines or -alkylamines or -alkyl phenylamines, their enantiomers or diastereoisomers and their pharmacologically acceptable salts.

There has thus been outlined, rather broadly, the more important features of the invention in order that the detailed description thereof that follows may be better understood and in order that the present contribution to the art may be better appreciated.

The invention may also include the preparation of pharmaceutical products with neuroprotective, anti-amnesic anticonvulsive, antidepressive and nooanaleptic-elevating vigilance and anti-fatigue actions for the first two entities and sedative-anxiolytic action for the last entities. There are, of course, additional features of the invention that will be described hereinafter and which will form the subject matter of the claims attached.

Numerous objects, features and advantages of the present invention will be readily apparent to those of ordinary skill in the art upon a reading of the following detailed description of present, but nonetheless illustrative, embodiments of the present invention. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a structural representation of 1-[4-[(1-Adamantyl)phenylmethyl]-1-phenyl]-4-methylpiperazine chloridrate, 1-[4-[(1-Adamantyl)phenylmethyl]-1-phenyl]-4-methylpiperazine bichloridrate, 1-[a-(1-Adamantyl)diphenylmethyl] 4-methylpiperazine bichloridrate, and 1-[4-[(1-Adamantyl)-4,4-diphenylbutl-1-yl]4-4-methylpiperazine bichloridrate.

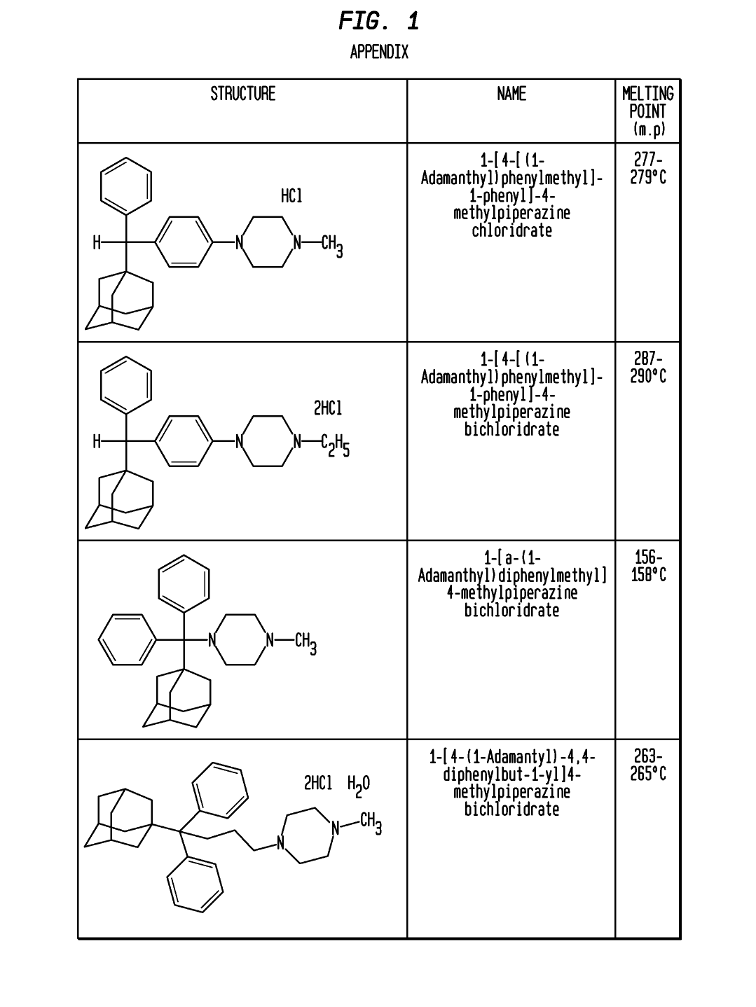

FIG. 2 is a structural representation of 1-[4-[(1-Adamantyl)-4,4-ethylpiperazine bichloridrate, 1-[4-[(1-Adamantyl)-4,4-diphenylbut-1-yl]-piperidine chloridrate, 1-[4-[(1-Adamantyl)-4,4-diphenylbut-1-yl]-4-cyclohexyl piperazine bichloridrate, and 4-[(1-Adamantyl)-N,N-dimethyl-4,4-diphenyl-1-butylamine chloridrate.

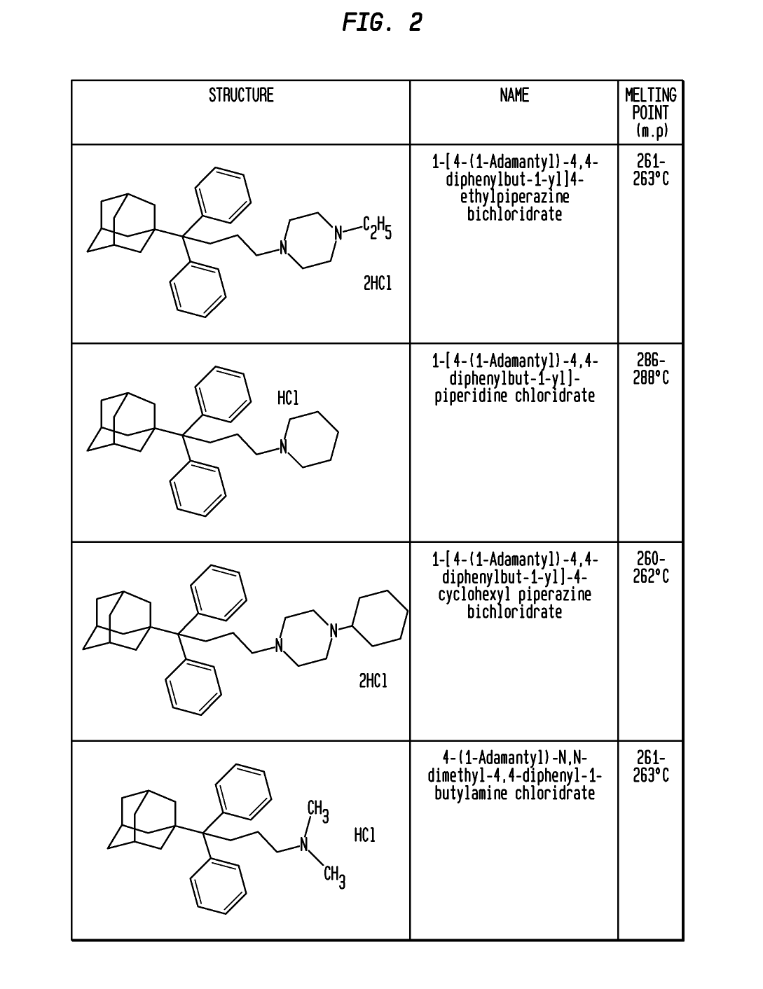

FIG. 3 is a structural representation of 1-[3-[4-[α-(1-Adamantyl)phenylmethyl-1-propyl]-4-methylpiperazine bichlordrate, 1-[5-(1-Adamantyl)-5-phenyl-1-pentyl] piperidinehydrochloride, 1-[5-(1Adamantyl)-5-phenyl-4-penten-1-yl]-4-methyl piperazine bishydrochloride, and 1-[5-(1-Adamantyl)-5-phenyl-1-pentyl]-4-methylpiperazinehydrochloride.

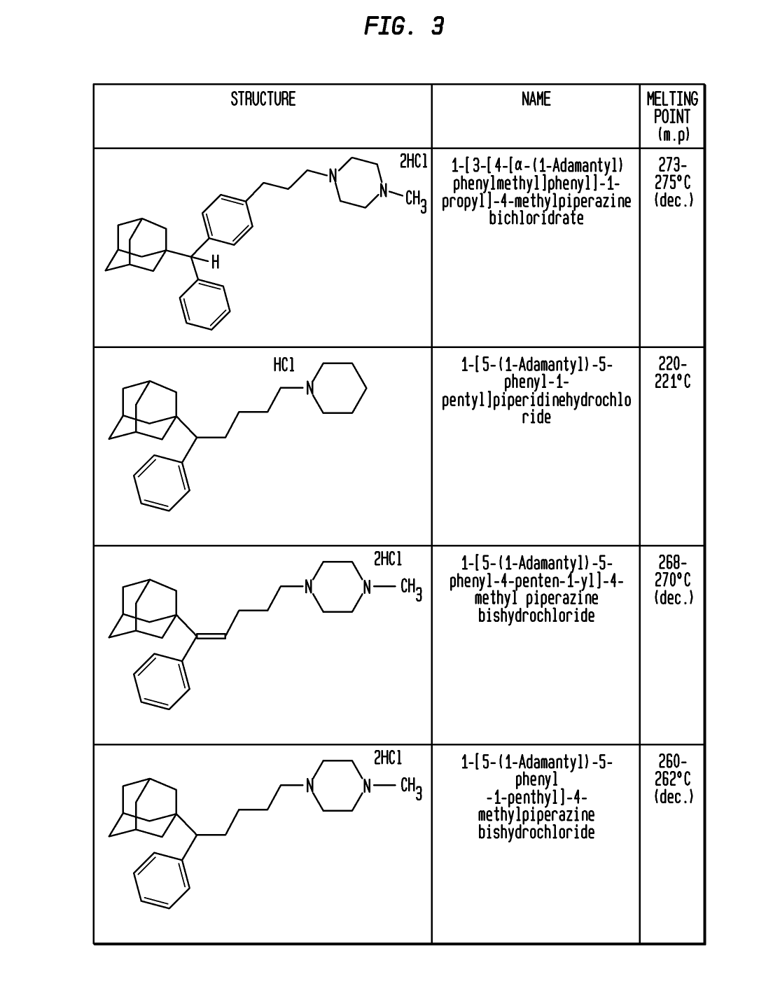

FIG. 4 is a structural representation of 1-[4-[(3-Cyclopenty-1-adamantyl)phenylmethyl-1-phenyl]-4-methylpiperazine hydrochloride, 1-[4-[3-Cyclopenty-1-adamantyl)phenylmethyl-1-phenyl]-4-ethylpiperazine bishydrochloride, -[4-[(3-Cyclopenty-1-adamantyl)phenylmethyl-1-phenyl]-4-cycloheylpiperazine hydrochloride, 3-Dimethylaminomethyl-5-(4-chlorophenoxy methyl)diphenuldihydrofuran-2(3H)-one hydrochloride.

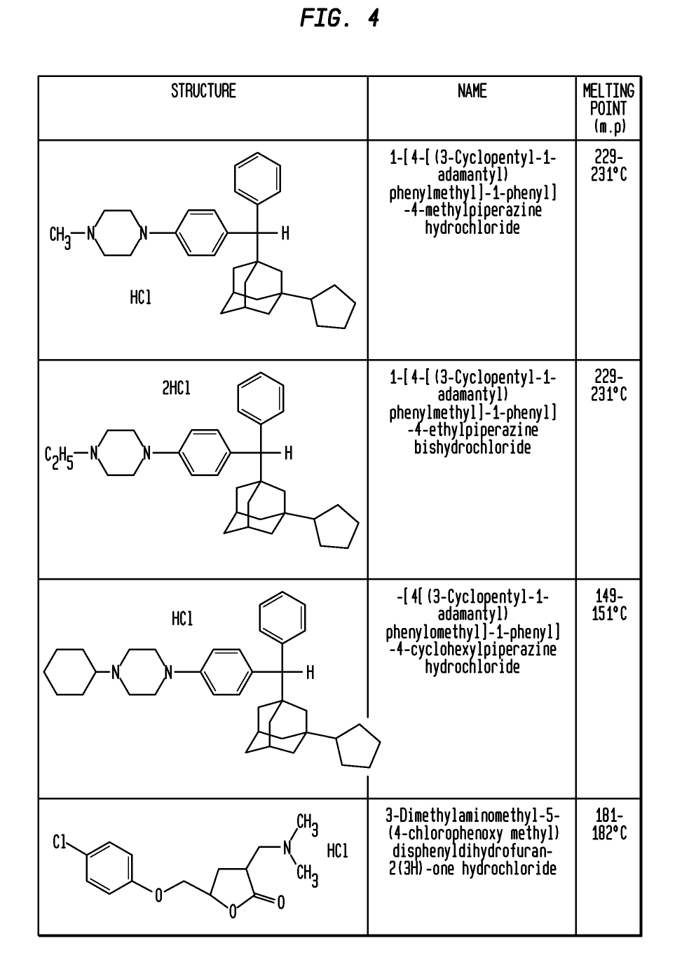

FIG. 5 is a structural representation of 5-(3-Cyclopentricyclo[3.3.1.1]dec-1-yl]-dihydro-3-(dimethylamino)-5-phenylfuran-2(3H)-one hydrochloride, 5-(Tricyclo[3.3.1.1]dec-1-yl]-dihydro-3-(diethylamino)-5-phenylfuran-2(3H)-one hydrochloride, 5-(Tricyclo[3.3.1.1]dec-1-yl]-dihydro-3-(1-piperdinomethyl)-5-phenylfuran-2(3H)-one hydrochloride, and 5-(Tricyclo[3.3.1.1]dec-1-yl]-dihydro-3-(4-methylpiperdazinomethyl)-5-phenylfuran-2(3)-one bishydrochloride.

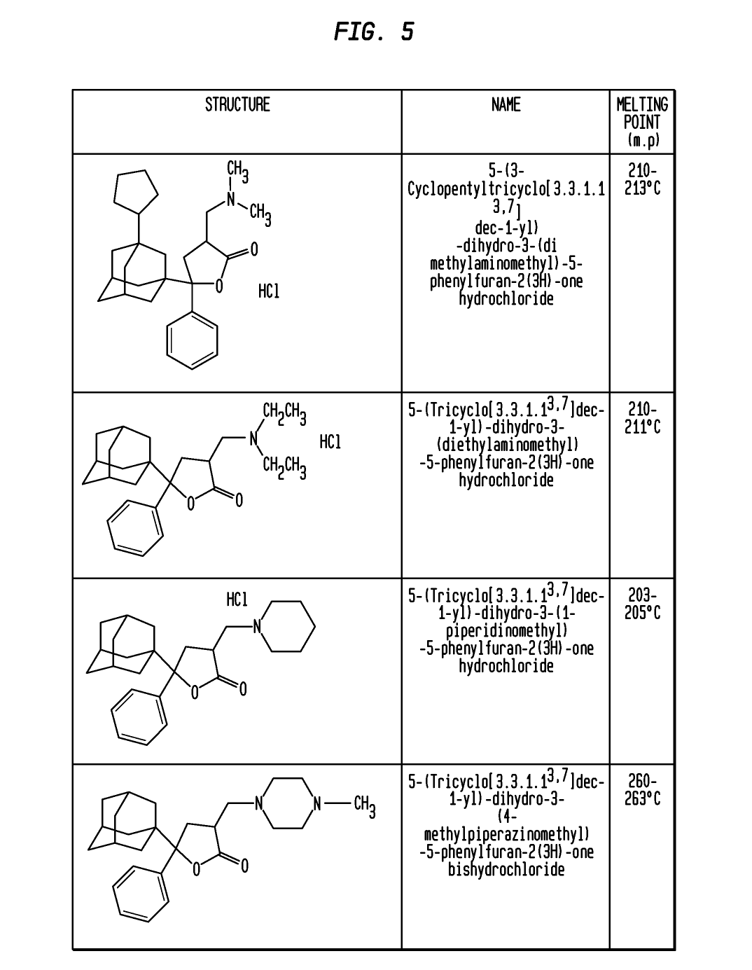

FIG. 6 is a structural representation of 5-(Tricyclo[3.3.1.1]dec-1-yl]-dihydro-3-(4-ethylpiperazinomethyl)-5-phenylfuran-2(3H)-one bishydrochloride, 5-(Tricyclo[3.3.1.1]dec-1-yl]-dihydro-3-(1-morpholinomethyl)-5-phenylfuran-2(3H)-one bishydrochloride, 5,5-Diphenly-N-[2-(1-phenylmethyl-4-piperidinyl)ethyl]tetrahydrofuran-3-carboxaminde chloridrate, and 5-(Tricyclo[3.3.1.1]dec-1-yl]-tertrahydro-Nmethyl-5phenyl-3-furanemethanamine hydrochloride.

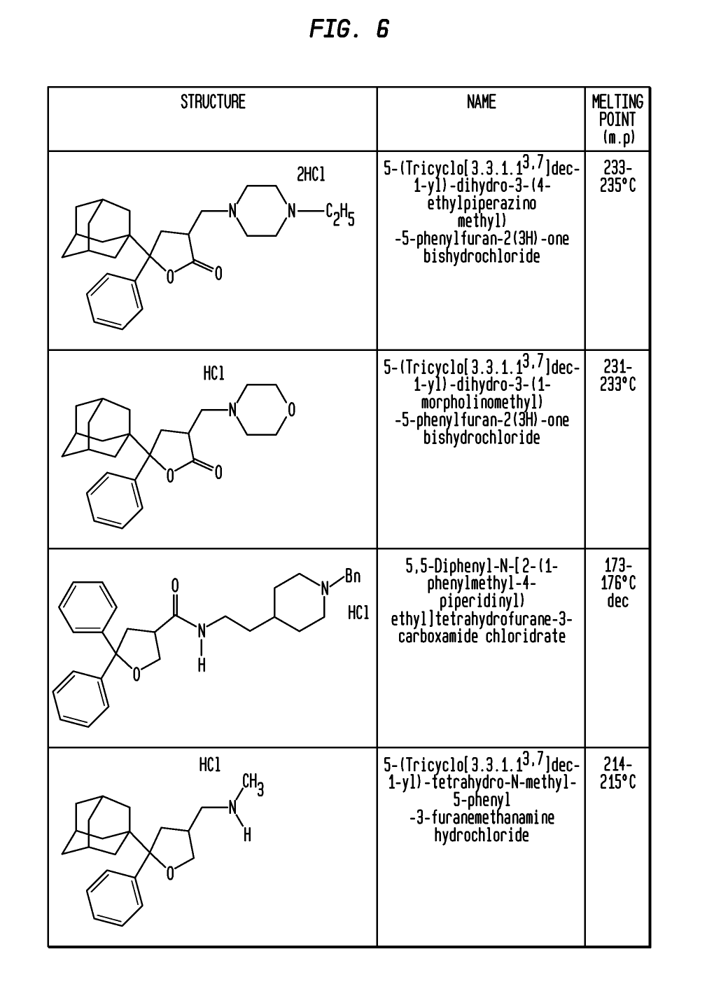

FIG. 7 is a structural representation of 5-(Cyclopentyltricyclo[3.3.1.1]dec-1-yl]-tetrahydro-N,N-dimethyl-5-phenyl-3-furanemethylamine hydrochloride.

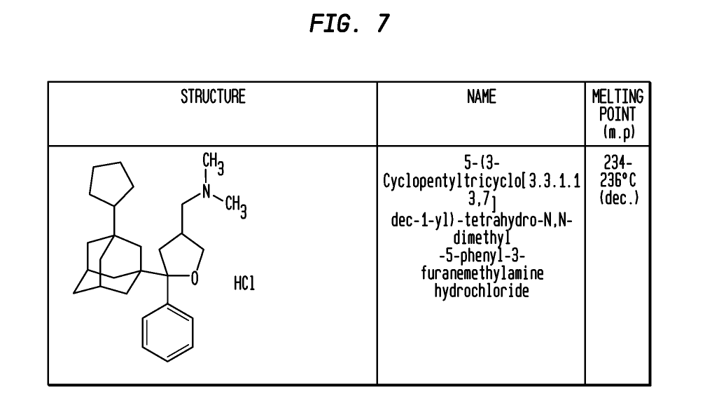

### DETAILED DESCRIPTION OF THE INVENTION

The present invention concerns the new and original (σ) sigma-receptor ligands (Mono- or Di-alkylaminoalkyl)-.gamma.-butyrolactones (AL), their analogues aminotetrahydrofuranes (AE), with various substitutes: phenyl-, diphenyl-, phenoxymethyl, fluorenyl or adamantyl, the (1-adamantyl)phenyl(s) alkylamines-(AdBAA), the N,N Dialkyl α[(adamantyl-1) benzyloxy-2] alkylamines-(AdBOAA) and the 3-cyclopentyl-adamantyl-amines or alkylamines or alkylphenyl amines (AdCP), halogenated, or methoxylated on their substitutes and their pharmaceutically acceptable salts of the racemics, enantiomers and diastereoisomers that were synthesized according to the methods described in the references: WO 97/30983, 1 and 2, with anti-cancer, anti-metastatic, anti-(chronic) inflammatory, neuroprotective, anticonvulsant, antidepressive, nooanaleptic and anxiolytic action.

The present invention concretize the concept of bio-modulatory drug, with the new and original σ-receptor ligands AL, AE, AdBAA, AdBOAA and AdCP, which exhibit a multi-component pharmacological profile with orthosteric, (σ-1 and/or σ-2-induced) allosteric, and (σ-1 and/or σ-2-induced) pseudo-allosteric modulatory actions over functional biochemical reactions.

In vitro and in vivo experiments clearly demonstrated the principal interaction of AL, AE, AdBAA, AdBOAA and AdCP over the σ-1, σ-2 receptors or over the allosteric σ-type sites at low nanomolar up to sub-micromolar levels of affinity along with their regulatory roles over the G-protein coupled receptors (GPCRs) and Na⁺/Ca⁺⁺ ion channels.

In vitro studies demonstrated the anti-cancerous properties of AL, AE, AdBAA, AdBOAA and AdCP which present σ-2 agonistic action and/or σ-1 antagonistic action via the mechanism of apoptosis which they specifically develop over the cancer cells.

The molecules mentioned above can be used as anti-cancer drugs, either alone or in combination with conventional anti-cancer drugs. They can also be used as diagnostics (sigma receptors are increasing in most of the different types of cancer cells) and as anti-metastatic drugs, since they inhibit the binding and migration of cancer cells via tumor cell membrane reorganization and inhibition of Na⁺ ion channels (particularly for AL and AE compounds).

Finally, their ability to regulate cellular apoptotic processes, through both membrane and mitochondrial σ-1 and/or σ-2 receptors, associated with their aptitude to inhibit auto-immune reactions confer at these molecules new opportunities to be used as therapeutic agents (and not only as symptomatic drugs, as previously claimed for some of them: AL, AE) against neurodegenerative diseases (σ-1 agonism and inhibition of Na⁺/Ca⁺⁺ ion channels) or against chronic inflammatory diseases (σ-1 antagonism, σ-2 agonism).

### EXAMPLES

A) Sigma (σ)-1 Agonists with Bio-Modulatory Action on the Central Nervous System (CNS) and with Antagonistic Action on the Apoptotic Processes of the Cells.

AE14: Tetrahydro-N,N,-dimethyl-5,5-diphenyl-3-furanomethanamine

AE14 is a selective ligand for σ-1 receptors with a nanomolar affinity (pKi=7.6) while the affinity for σ-2 receptors is more than 100 times lower [over 10 micro-Moles (10 [symbol] M): pKi<5]. The above molecular profile is the basis for AE14 modulatory role over the G-protein coupled receptors (GPCRs) and its pharmacological properties: anti-amnesic action (antagonistic action against scopolamine and dizocilpine (MK-801) induced amnesia or amyloid peptide β25-35 (Aβ25-35), at low doses (from 0.03 mg/kg administered per os (po), in mice). This unique new profile of AE14: anti-apoptotic via agonistic sigma-1 regulation of the volume regulated chloride channels (VRCC), anti-oxidative stress via agonistic action on the sigma-1 receptors of the mitochondrion and the reticulum endoplasmic (ER), anti-microinflammatory effect via sigma-1 modulation of the micro-astroglial hyper-reactivity and modulation of the GPCRs, in the central nervous system (CNS), confer at this molecule putative therapeutic—and not only symptomatic, as previously claimed,—pharmaceutical applications against neurodegenerative diseases, especially Alzheimer's disease but also multiple sclerosis, Parkinson's disease and all forms of neurodegenerations coming from the above mentioned neuronal dysregulations.

Due to its modulatory role via σ-1 and, at concentrations over 10 [symbol] M, also via σ-2 receptors and Na⁺channels, AE14 presents strong antidepressive profile (very strong anti-immobility action at 3σ-100 mg/kg (po), in the forced swimming test in mice and useful nooanaleptic action (non addictive since neurochemical studies in rats excluded dopaminegic action), anti-fatigue and antidepressive properties, according to the relevant experimental protocols in mice (Open field, Porsolt).

The cytotoxic action of AE 14 on the cancer cells, via the mechanism of apoptosis, is shown only at high concentrations (over 200 [symbol] M, depending on the cell line that it is used), since AE14 partial agonistic action for σ-1 is transformed in these concentrations to antagonistic σ-1 action in synergy with its agonistic action for σ-2 that is shown at concentrations over 10 [symbol] M. These results are representatives of the basic concept of the invention described, (see the last two paragraphs above). AE14 presents anti-apoptotic action (neuroprotective and anti-amnesic action against neurodegenerative diseases at low σ-1 agonistic doses) and pro-apoptotic action for σ-1 antagonistic and σ-2 agonistic high doses (200 [symbol] M equivalent to 200 mg/kg per os in mice) with anti-cancer properties and action against chronic inflammatory diseases.

In regards to the last action (pro-apoptotic), the presence of adverse events at high concentrations of AE14, led us to the discovery or synthesis of other compounds that were more effective and without adverse events (see section B).

AE37: tetrahydro-N,N-dimethyl-2,2-diphenyl-3-furanomethanamine

Similar pharmacological profile as to AE14: smaller affinity for σ-1 receptors (pKi=6.3) and with no affinity for σ-2 receptors.

Therefore, AE37, as a selective σ-1 agonist with anti-apoptotic action, is especially a new putative therapeutic—and not only symptomatic, as previously claimed—agent agent against neurodegenerative diseases, very performant (0.03-1 mg/kg, i.p) against the Aβ25-35 induced neutotoxicity and oxidative stress. AE37 is also an original anticonvulsive agent (via its σ-1 modulatory action over the Na⁺ and Ca⁺⁺ ion channels and its weak anti-glutamatergic action in the brain). Indeed, in contrast to the clinically used antiepileptic drugs—which were recently incriminated to be pro-apoptotic and, therefore, amplifiers of the neuronal losses coming from the epileptic crises—AE37, in good agreement with its σ-1 agonism, was recently confirmed as anti-apoptotic and, therefore, as a new prototypical putative anti-epileptic drug protective against the neuronal losses coming from the epileptic crises, in monotherapy and also active against the neuronal losses coming from the pro-apoptotic effects of the clinically used anti-epileptics, when AE37 could be clinically associated with the above anti-epileptics. The above new prototypical antiepileptic and neuroprotective profile of AE37 is completed by the prementioned CNS indications of AE14 (vigilance, anti-fatigue and putative antidepressive).

AE37 Met: tetrahydro-N-methyl-2,2-diphenyl-3-furanomethanamine

AE37 Met is the only metabolite of AE37 and is slightly more active than AE37 for the properties mentioned above but it mainly concerns the neuroprotective action, especially against acute ischemic syndromes (brain, heart), in relevance with its exceptional protection against anoxia that is caused by pentylenetetrazole (PTZ), in mice, following its protection against the PTZ-induced tonic crises.

B) Weak 6-1 Agonists or σ-1 Antagonists with Agonistic σ-2 Component and Agonistic Action to the Apoptotic Processes of the Cells.

The following molecules are representatives of the chemical families that concern the present invention:

1) AdAL: DIHYDRO-4-(DIMETHYLAMINOMETHYL)SPIRO{FURANS-2(5H), 2′-TRICYCLO[3.3.1.1_{3,7}]DECAN}-5-ONE 6

2) AdAE: (DIHYDRO-4-(DIMETHYLAMINOMETHYL)SPIRO{FURANE-2(3H), 2′TRICYCLO[3.3.1.1_{3,7}]DECANE}

3) AdPhAL: 5-(TRICYCLO[3.3.1.1_{3,7}]DEC-1-YL)-DIHYDRO-3-DIMETHYLAMINOMETHYL)-5-PH-ENYLFURAN-2(3H)-ONE-

4) AdPhAE: 5-(TRICYCLO[3.3.1.1_{3,7}]DEC-1-YL)-TETRAHYDRO-N,N-DIMETHYL-5-PHENYL-3-FURANEMETHANAMINE-

5) AL142Me: (α-(DIMETHYLAMINOETHYL)-_{γγ}-DIPHENYL_{γ}-BUTYROLACTONE)

6) AdBPA: _{γ}-(1-ADAMANTYL)PHENYL-N,N-DIMETHYLPROPANAMINE

7) AdBPP: _{γ}-(1-ADAMANTYL)PHENYL-PROPYLPIPERIDINE

8) AdBOPP: [α-(ADAMANTYL-1)BENZYLOXY-2]PROPYLPIPERIDINE

9) AdBOEA (Me): N,N DIMETHYL-[α-(ADAMANTYL-1)BENZYDRYLOXY-2-ETHYLAMINE

10)-Ad(Me)CP: 3-CYCLOPENTYL-N-METHYL-1-TRICYCLO[3.3.1.1_{3,7}]DECANEMETHANAMINE

(See also, in appendix, undescribed derivatives of the above chemical families)

All molecules present strong chemical affinities (nanomolar) for σ-1 receptors with antagonistic action (except AdAE and AdPhAE which are weak agonists) and with nanomolar, sub-micromolar or micromolar affinity for σ-2 receptors with agonistic action. Therefore, all molecules above and their derivatives are pro-apoptotic and only at very low concentrations some of them (AdAE, AdPhAE) are anti-apoptotic. All molecules above are toxic to the cancer cells and make excellent drug candidates due to their exceptional and selective pro-apoptotic properties and their very low toxicity in vivo and therefore they could also be used against chronic inflammatory diseases (Crohn's disease, rheumatoid arthritis, chronic obstructive pulmonary disease and microinflammations of the nervous system in neurodegenerative diseases). It has been shown that at the concentrations and doses (1σ-100 mg/kg intra-peritoneal (ip) or po), at which anti-cancer action is demonstrated, most of the above representative molecules of AL, AE, AdBAA, AdBOAA, and AdCP do not present toxic events but, in contrast, they present positive properties such as: neuroprotective, anti-depressive, elevating vigilance, nooanaleptic and anti-fatigue (against fatigue syndrome), for AL, and AE, or sedative-anxiolytic properties for AdBAA, AdBOAA and AdCP according to the relevant experimental protocols in mice.

### REFERENCES

1) Pouli N., Fytas G., Foscolos G., Kolocouris N., Marakos P., and Vamvakides A. Synthese et etude pharmacologique des adaman-tylbenzenepropanamines et propenamines. Annales Pharmaceutiques Francaises, 1995, 53, No 4, pp 163-169

2) Fytas G., Marakos P., Kolocouris N., Foscolos G., Pouli N., Vamvakides A., Ikeda S., De Clercq E. 3-cyclopentyl-1-adamantamines and adamantanemethanamines. Antiviral activity evaluation and convulsion studies. Farmaco 1994, 49, No 10, pp 641-647

3) Vamvakides Alexandre: Tetrahydro-N,N-dimethyl-2,2-diphenyl-3-furanemethanamine, its enantiomers and their pharmaceutically acceptable acid addition salts. WO97/30983 and patent 1002616 (GR)

1. A sigma(σ)-receptor ligand with anti-apoptotic and/or pro-apoptotic properties said compound consisting essentially of:
a sigma(σ)-receptor ligand compound consisting essentially of tetrahydro-N,N-dimethyl-2,2-diphenyl-3-furanomethanamine, enantiomers or diastereoisomers of tetrahydro-N,N-dimethyl-2,2-diphenyl-3-furanomethanamine, and pharmacologically acceptable salts of tetrahydro-N,N-dimethyl-2,2-diphenyl-3-furanomethanamine.

2-4. (canceled)

5. The compound according to claim 1 wherein said tetrahydro-N,N-dimethyl-2,2-diphenyl-3-furanomethanamine is a metabolite of said tetrahydro-N,N-dimethyl-2,2-diphenyl-3-furanomethanamine.

6. The compound of claim 5 wherein said metabolite is tetrahydro-N-methyl-2,2-diphenyl-3-furanomethanamine
